(19) United States
(12) Patent Application Publication
(10) Pub. No.: US 2022/0257493 A1
(43) Pub. Date: Aug. 18, 2022

(54) NAIL TREATMENT COMPOSITION

(71) Applicant: L'OREAL, Paris (FR)

(72) Inventors: Ronak RUGHANI, EDISON, NJ (US); Ramakrishnan HARIHARAN, SPRINGFIELD, NJ (US); Sam MORRISON, CLARK, NJ (US); I-Fan HSIEH, SCOTCH PLAINS, NJ (US)

(73) Assignee: L'OREAL, Paris (FR)

(21) Appl. No.: 17/174,389

(22) Filed: Feb. 12, 2021

(51) Int. Cl. A61K 8/73; A61K 8/46; A61K 8/42; A61K 8/365; A61Q 3/02

(52) U.S. Cl. CPC A61K 8/731; A61K 8/46; A61Q 3/02; A61K 8/365; A61K 8/42

(57) ABSTRACT

Cosmetic compositions for treating the nails include a water-insoluble cellulose polymer, one or more additional film-formers, plasticizer, and a urea compound. Methods of using the compounds to treat nails are also provided.

### FIELD OF THE INVENTION

The present invention relates to nail treatment compositions, and specifically to nail treatment compositions including urea compounds to provide benefits including nail strengthening and surface smoothening.

### BACKGROUND

Consumers use nail enamel to cosmetically enhance the appearance of their nails (fingernails and/or toenails) or protect the nails from the abuses found in their everyday environment. Often this requires a nail enamel that is formulated to provide a good shine and/or color.

The inventors have recognized that, aside from shine and color, it is also desirable to provide other benefits such as hardness and/or surface smoothness to the nail, particularly hardness and/or surface smoothness that lasts after the nail enamel is removed.

A nail treatment composition providing lasting benefits to the nail is therefore highly desirable.

### BRIEF SUMMARY

According to one aspect of the invention, a cosmetic composition for treating the nails, includes a water-insoluble cellulose polymer; one or more additional film-formers; plasticizer; and a urea compound. The urea compound is free of both urethane groups and of cyclic groups. The cosmetic composition as a whole is free of urea compounds having either or both of urethane groups and cyclic groups. Further, the cosmetic composition is anhydrous.

According to another aspect of the invention, the cosmetic composition includes from about 5% by weight to about 20% by weight of nitrocellulose; from about 5% by weight to about 20% by weight of one or more additional film-formers; plasticizer; from about 50% to about 90% by weight of an acetate-rich solvent system; and a urea compound. The urea compound is free of both urethane groups and of cyclic groups. The nitrocellulose and the one or more one or more additional film-formers form a film-forming system, and the ratio by weight of the urea compound free of both urethane groups and of cyclic groups to the film-forming system is from about 1:500 to about 1:50. The cosmetic composition as a whole is free of urea compounds having either or both of urethane groups and cyclic groups. Further, the cosmetic composition is anhydrous.

According to another aspect of the invention, the cosmetic composition includes a water-insoluble cellulose polymer; one or more additional film-formers; plasticizer; a urea compound; and one or more organic polycarboxylic acids. The cosmetic composition is anhydrous.

According to another aspect of the invention, a method of treating the nails is provided. The method includes applying to the nails a cosmetic composition. The cosmetic composition includes a water-insoluble cellulose polymer; one or more additional film-formers; plasticizer; and a urea compound free of both urethane groups and of cyclic groups. The cosmetic composition as a whole is free of urea compounds having either or both of urethane groups and cyclic groups. Further the cosmetic composition is anhydrous. The method may further include removing the cosmetic composition, and optionally reapplying and removing the composition one or more times to effect a reduction in surface roughness in the nail of greater than 10%.

### DETAILED DESCRIPTION

As used herein, articles such as “a” and “an” when used in a claim, are understood to mean one or more of what is claimed or described.

As used herein, the expression “at least one” means one or more and thus includes individual components as well as mixtures/combinations.

All concentrations/percentages listed are by weight unless otherwise noted. Numerical ranges are inclusive of endpoints and meant to include all combinations and sub-combinations. For example, from about 5%, 10% or 15% to about 20%, 50% or 60% means: about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%.

Other than in the operating examples, or where otherwise indicated, all numbers expressing quantities of ingredients and/or reaction conditions are to be understood as being modified in all instances by the term “about,” meaning within 15%, such as within 10%, of the indicated number. For example, about 10% means from 8.5% to 11.5%, such as between 9% and 11%.

“Actives basis” as used herein means considering only the particular component of ingredient (e.g., in a composition) and ignoring other chemically unrelated components that may be also be present in the same raw material source of that particular component.

“Polymer” as used herein means a compound which is made up of at least two monomers.

“Substantially free” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. In certain embodiments, substantially free means less than about 2% of the identified ingredient, such as less than about 1%, such as less than about 0.5%, such as less than about 0.25% of the ingredient, such as less than about 0.1% of the ingredient. In certain embodiments, compositions of the present invention are anhydrous, meaning substantially free of water. In certain other embodiments, compositions of the present invention are substantially free of one, more or all of ammonium salts, lactic acid or lactates.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, hydroxyalkyl groups, and polysiloxane groups. The substituent(s) may be further substituted.

“Volatile”, as used herein, means having a flash point of less than about 50° C.

As used herein, the terms “include”, “includes” and “including” are meant to be non-limiting.

### Urea Compounds

The present invention is directed to a nail compositions including one or more urea compounds. By “urea compounds,” it is meant a compound including a urea (carbamide) group, i.e., two amide groups joined by a carbonyl group [—NH—(C═O)—NH-].

The urea compounds may have a structure in accordance with the following formula:

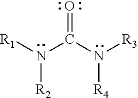

wherein R1, R2, R3, and R4 are independently selected from hydrogens, C4 to C10 unsubstituted aryl, C₄ to C₁₀ substituted aryl, C₂ to C₁₀ unsubstituted heterocycle, C₂ to C₁₀ substituted heterocycle, C₁ to C₁₀ unsubstituted alkyl, C₁ to C₁₀ substituted alkyl, C₃-C₁₀ unsubstituted cycloalkyl, and C₃-C₁₀ substituted cycloalkyl.

Non-limiting examples of urea compounds include urea, urea derivatives, imidazolidinyl urea, diazolidinyl urea, m-dimethylaminophenyl urea, dimethyl urea, a hydroxyethyl urea, N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-(tris-hydroxymethylmethyl)urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′, and N′-bis(2-hydroxypropyl)-urea.

According to certain embodiments, the one or more urea compounds include a urea compound that is free of both urethane groups and free of cyclic groups. By “urea compound free of both urethane groups and of cyclic groups,” it is meant that the particular urea compound has no urethane linkages [O—C═O—NH-] and also has no cyclic (homocyclic or heterocyclic—of aliphatic or aromatic nature) groups. According to certain embodiments, the urea compound free of both urethane groups and of cyclic groups includes at least one, such as exactly one hydroalkyl group. According to certain other embodiments, the hydroxyalkyl group has one to eight carbon atoms. According to certain other embodiments, the urea compound that is free of both urethane groups and free of cyclic groups is water-soluble, e.g., may dissolve in water at standard conditions at least about 10% by weight.

According to certain notable embodiments, the urea compound free of both urethane groups and of cyclic groups is selected from urea, hydroxyethylurea (C₃H₈N₂O₂), such as 2-hydroxyethylurea, and dimethylurea. In certain embodiments, the urea compound is hydroxyethylurea.

The concentration by weight of the urea compound (e.g., one free of both urethane groups and of cyclic groups) in the composition may range from about 0.025%, 0.1%. 0.15% or 0.2% by weight to about 0.25%, 0.5%, 1%, 5% or 10% by weight.

According to certain embodiments, the cosmetic composition in its entirety is free of or is substantially free of urea compounds having either or both of urethane groups and cyclic groups. By “free of urea compounds having either or both of urethane groups and cyclic groups,” it is meant the composition does not include any of the following: (1) urea compounds that have urethane groups, (2) urea compounds that have cyclic groups; (3) urea compounds that have both urethane groups and cyclic groups.

### Organic Polycarboxylic Acid

Compositions of the present invention optionally include organic polycarboxylic acids. The organic polycarboxylic acids may be an aliphatic saturated polycarboxylic acids such as oxalic, malonic, succinic, malic, tartaric, citric, adipic acid or aliphatic unsaturated polycarboxylic acids such as fumaric or maleic acid. In a particular embodiment, the organic polycarboxylic acid is saturated and may have four to six carbon atoms. In a particularly notable embodiment, the one or more organic polycarboxylic acids includes citric acid. In other embodiment, citric acid is the only aliphatic polycarboxylic acid in the composition.

The concentration by weight of the organic polycarboxylic acids in the composition may range from about 0.025%, 0.1%. 0.15% or 0.2% by weight to about 0.25%, 0.5%, 1%, 5% or 10% by weight.

In certain embodiments, the nail composition may be formulated to have a weight ratio of the organic polycarboxylic acid (i) to the urea compound of (ii) may be about 10:1 to about 2:10. In some instances, the cosmetic composition may be formulated to have a weight ratio of citric acid to urea compound(s) of [about 10:1 or 9:1 or 8:1 or 7:1 or 6:1 or 5:1 or 4:1 or 3:1] to [about 2:3 or about 2:4 or about 2:5 or about 2:6 or about 2:8 or about 2:10].

### Water-Insoluble Cellulose Polymer

According to embodiments of the present invention, the nail composition also includes a water-insoluble cellulose polymer, which serves as a film-former.

The film former may comprise a cellulose derivative. Suitable water-insoluble cellulose polymers include cellulose or chemically modified cellulose such as those obtained by substituting a part or all of the hydroxyl groups in the cellulose with other groups. The water-insoluble cellulose polymers may be selected from cellulose, nitrocellulose, cellulose acetate, cellulose acetate phthalate, hydroxypropylmethylcellulose phthalate, and combinations thereof. According to certain embodiments, the water-insoluble cellulose polymer is nitrocellulose.

The concentration by weight of the water-insoluble cellulose polymer in the composition may range from about 1, 2%, 3% or 5% by weight to about 5%, 7%, 8%, 10%, 12%, 15%, 20% or 25% by weight.

### Additional Film Formers

According to embodiments of the present invention, the nail composition also includes additional film-formers. These are typically polymers that complement or enhance the film forming performance of the water-insoluble cellulose.

In certain embodiments, the film former may comprise an acrylic polymer (homopolymer or copolymer). Specific examples of suitable primary film formers include, but are not limited to, synthetic polymers of the polycondensate type or of the free-radical type, acrylic polymers resulting from the copolymerization of monomers chosen from the esters and/or amides of acrylic acid and/or of methacrylic acid (as examples of monomers of ester type, mention may be made of methyl methacrylate, ethyl methacrylate, butyl methacrylate, isobutyl methacrylate, 2-ethylhexyl methacrylate and lauryl methacrylate. As examples of monomers of amide type, mention may be made of N-t-butylacrylamide and N-t-octylacrylamide), and acrylic polymers obtained by copolymerization of ethylenically unsaturated monomers containing hydrophilic groups, preferably of nonionic nature, such as hydroxyethyl acrylate, 2-hydroxypropyl acrylate, hydroxyethyl methacrylate and 2-hydroxypropyl methacrylate.

In certain embodiments, the film forming polymer may comprise (meth)acrylate homopolymers and copolymers, polyurethanes, polyacryls, polymethacryls, cellulosic polymers, styrene-acryl copolymers, polystyrene-polyacryl mixtures, polysiloxanes, polyethers, polyesters, urethane-acryl copolymers, cellulose acetate propionate, siloxane-urethane copolymers, polyurethane-polymethacryl mixtures, silicone-acryl copolymers, vinyl acetate polymers, and mixtures thereof.

The film forming polymer may comprise aldehyde condensation products such as arylsulfonamide formaldehyde resins, specifically toluene sulfonamide formaldehyde resin which is a condensation product of formaldehyde and toluene sulfonamide, toluene sulfonamide/epoxy resins, e.g. tosylamide and non-drying alkyd resins, acrylic polymers and copolymers, polyurethane, polyacryls, polymethacryls, styrene-acryl copolymers, polystyrene-polyacryl mixtures, polysiloxanes, polyethers, polyesters, urethane-acryl copolymers, siloxane-urethane copolymers, polyurethane-polymethacryl mixtures, vinyl acetate polymers, and mixtures thereof.

In certain embodiments, the film forming polymer may be a polymer comprising repeating units of at least one (meth)acrylate unit and vinyl copolymers. Potential acrylates copolymers include, but are not limited to, those sold under the PECOREZ® name such as, for example, PECOREZ AC 50.

In certain embodiments, the film forming polymer may be a slightly branched, hydroxyl-bearing polyester. Potential slightly branched, hydroxyl-bearing polyesters for use in the present invention include, but are not limited to, those sold under the BAYCUSAN® name such as, for example, BAYCUSAN® XP 8000.

In certain embodiments, the film forming polymer may be a polyurethane in solvents, having between about 20% and about 70% solids content. Certain embodiments may be aliphatic resins. Potential urethanes for use in the present invention include, but are not limited to, those sold under the REZE-LASTIC™ or REZE-MERIC™ name such as, for example, REZE-LASTIC™ 2140, REZE-MERIC™ 3182, or REZE-MERIC™ 3197.

The film forming polymer may comprise polyester resins formed by reacting a polyhydric alcohol with a polybasic acid, e.g., phthalic acid, such as the commercial product sold by Unitex Chemical Corporation under the name UNIPLEX 670-P, which is a polyester resin obtained by reacting trimellitic acid, neopentyl glycol, and adipic acid. (Meth)acrylic resins according to the disclosure may include copolymers of methyl methacrylate with butyl acrylate, butyl methacrylate, isobutyl methacrylate, or isobornyl methacrylate, for example, the commercial products PARALOID DM-55, PARALOID B48N, PARALOID B66, and ELVACITE 2550; copolymers of isobutylmethacrylate and butyl methacrylate, for instance, the commercial product ELVACITE 2046; and isobutyl methacrylate polymers, for example, PARALOID B67.

In some embodiments, at least one film forming polymer is selected from carboxyl functional acrylate polymers having a moderate to high glass transmission temperature (Tg) value. The “moderate to high glass transmission” as used in the context of the present invention, refers to an acrylates copolymer having Tg value ranging from about 40° C. to about 95° C., more preferably from about 60° C. to about 90° C., and more preferably from about 70° C. to about 85° C. In some embodiments, at least one film forming polymer has a relatively high acid value, such as for example an acid value (number) ranging from about 40 to about 95, preferably from about 50 to about 90, and preferably from about 60 to about 80, including all ranges and subranges therebetween. Commercially available examples of these film forming polymers include but are not limited to, for example, those sold under the ISOCRYL® name by Estron Chemicals such as, for example, Isocryl C-70 (styrene/acrylates copolymer), Isocryl N-2513, Isocryl H-60, and Isocryl H-1871.

The film forming polymer may comprise an epoxy resin. In some embodiments, the epoxy resin has a glass transition temperature (Tg) of less than about 100° C., preferably less than about 80° C. Non-limiting examples of suitable epoxy resins include tosylamide epoxy resins, such as those sold by Estron Chemical under the tradename Polytex™, e.g., E-75, E-100, and NX-55, NX-3214). Other non-limiting examples of suitable epoxy resins include aryl-sulfonamide epoxy resins.

In certain embodiments, the film former comprises a silicone-organic polymer hybrid compound.

In some embodiments, the silicone-organic polymer may comprise a silicone acrylate copolymer, which may have a glass transition temperature (Tg) of greater than 20° C. Suitable examples of silicone acrylate copolymers include silicone/(meth)acrylate copolymers. Suitable examples also include polymers derived from non-polar silicone copolymers comprising repeating units of at least one polar (meth)acrylate unit and vinyl copolymers grafted with at least one non-polar silicone chain. Non-limiting examples of such copolymers are acrylates/dimethicone copolymers such as those commercially available from Shin-Etsu, for example, the products sold under the tradenames KP-545 (cyclopentasiloxane (and) acrylates/dimethicone copolymer), KP-543 (butyl acetate (and) acrylates/dimethicone copolymer), KP-549 (methyl trimethicone (and) acrylates/dimethicone copolymer), KP-550 (tentative INCI name: isododecane (and) acrylate/dimethicone copolymer), and mixtures thereof. Additional examples include the acrylate/dimethicone copolymers sold by Dow Corning under the tradenames FA 4001 CM SILICONE ACRYLATE (cyclopentasiloxane (and) acrylates polytrimethylsiloxymethacrylate copolymer) and FA 4002 ID SILICONE ACRYLATE (isododecane (and) acrylates polytrimethylsiloxymethacrylate Copolymer), and mixtures thereof. Suitable examples also include polymers comprising a backbone chosen from vinyl polymers, methacrylic polymers, and acrylic polymers, and at least one chain chosen from pendant siloxane groups. These polymers may be sourced from various companies. One such company is Minnesota Mining and Manufacturing Company which offers these types of polymers under the tradenames “Silicone Plus” polymers (for example, poly(isobutyl methacrylate-co-methyl FOSEA)-g-poly(dimethylsiloxane), sold under the tradename SA 70-5 IBMMF). Suitable examples also include silicone/acrylate graft terpolymers, for example, the copolymers described in WO 01/32727 A1, the disclosure of which is hereby incorporated by reference. Suitable examples also include polymers comprises a backbone chosen from vinyl backbones, methacrylic backbones, and acrylic polymeric backbones and further comprises at least one pendant siloxane group. A non-limiting example of these polymers is poly(dimethylsiloxane)-g-poly(isobutyl methacrylate), which is commercially available from 3M Company under the tradename VS 70 IBM.

The at least one silicone-organic polymer hybrid compound may be a silicone vinyl acetate compound and/or a crosslinked anionic copolymer comprised of organic polymer blocks and silicone blocks, resulting in a multiblock polymer structure, or mixtures thereof. In particular, suitable examples of the silicone-organic polymer hybrid compound of the present invention include, but are not limited to, copolymers of vinyl acetate and vinyl silicone. Such copolymers may optionally further include C3-7 carboxylic acid and/or vinyl C7-45 alkyl ester monomers. Suitable examples of the silicone-organic polymer hybrid compound of the present invention include, but are not limited to, crosslinked anionic copolymers comprising at least one cross-linked polysiloxane structural unit. Specific examples include a compound having the INCI name of Crotonic Acid/Vinyl C8-12 Isoalkyl Esters/VA/Bis-Vinyldimethicone Crosspolymer which is a copolymer of Crotonic Acid, vinyl C8-12 isoalkyl esters and Vinyl Acetate crosslinked with bis-vinyldimethicone. This compound is commercially available from the company Wacker Chemie AG under the tradename WACKER BELS IL® P1101 (may also be known under the tradename WACKER BELSIL ® P101). Crotonic Acid/Vinyl C8-12 Isoalkyl Esters/VA/Bis-Vinyldimethicone Crosspolymer is also known by the technical name of Crotonic Acid/Vinyl C8-12 Isoalkyl Esters/VA/divinyldimethicone Crosspolymer.

In certain embodiments, the film former comprises an alkyd resin. Preferably, the at least one alkyd resin is a polyester comprising hydrocarbon chains of fatty acids. Such resins can be obtained by polymerization of polyols and polyacids or their corresponding anhydride in the presence of fatty acids, where the fatty acids can be employed “as is” or in the form of fatty acid triglycerides or in the form of oils during the synthesis of the alkyd resin. Examples of suitable fatty acids which can be employed in the synthesis of alkyd resins include, but are not limited to, at least one of fatty acids corresponding to the formula R—COOH, in which R denotes a saturated or unsaturated hydrocarbon radical preferably having from 7 to 45 carbon atoms, preferably from 9 to 35 carbon atoms, preferably from 15 to 35 carbon atoms and preferably from 15 to 21 carbon atoms. Mention may be made of, for example, palmitic acid, stearic acid, oleic acid, ricinoleic acid, linoleic acid, linolenic acid and, in particular, capric acid. Specific examples of acceptable alkyd resins include, but are not limited to, at least one of those sold under the names “Beckosol ODE 230 70E” by Dainippon Ink & Chem (phthalic anhydride/glycerol/glycidyl decanoate copolymer in ethyl acetate at 70%), “NECOWEL 581®” (50% in soybean oil), “NECOWEL 585®” (20% in sunflower oil), “NECOWEL 580®” (20% in sunflower oil), “NECOWEL 586 N®” (50% in soybean oil), “NECOWEL EP 1161®” (50% in soybean oil), “NECOWEL EP 1213®” (20% in oil), “NECOWEL EP 2009®” (32% in sunflower oil), “NECOWEL EP 2019®” (20% in oil), “NECOWEL EP 22750” (35% in oil), “NECOWEL EP 2329®” (34% in oil), and “NECOWEL EP 3016®” (30% in oil) by Ashland or “URADIL XP 515 AZ®” (73% in tall oil) or “URADEL XP 516 AZ®” (63% in tall oil) by DSM Resins. Beckosol ODE 230 70E (phthalic anhydride/glycerol/glycidyl decanoate copolymer in ethyl acetate at 70%) is particularly preferred.

The concentration by weight of the additional film-formers in the composition may range from about 1, 2%, 5% or 10% by weight to about 5%, 7%, 8%, 10%, 12%, 15% or 20% by weight.

According to certain embodiments the combined concentration by weight of the water-insoluble cellulose polymer and the additional film former is from about 15% or 20% to about 25%, or 30%, or 40% by weight. According to certain other embodiments, the water-insoluble cellulose polymer is present in a concentration by weight that is at least 30% of the sum of the concentrations by weight of the water-insoluble cellulose polymer and the additional film formers.

According to certain embodiments, the water-insoluble cellulose polymer (such as nitrocellulose) and the one or more one or more additional film-formers form a film-forming system and the ratio by weight of the urea compound free of both urethane groups and of cyclic groups to the film-forming system is from about 1:500 or 1:400 to about 1:300 or 1:200 or 1:100 or 1:50.

### Plasticizer

According to embodiments of the present invention, the nail composition also includes at least one plasticizer. The plasticizer is generally capable of plasticizing cellulose compounds such as nitrocellulose and/or the additional film-formers. The plasticizer may be selected from the group consisting of a benzoic acid derivative, a citric acid derivative, and an isobutyrate derivative. More preferably, the plasticizer is a benzoic acid derivative and/or a citric acid derivative.

In certain embodiments, the benzoic acid derivative may be a dibenzoate ester, including but not limited to diethylene glycol dibenzoate, dipropylene glycol dibenzoate, and 1,2-propylene glycol dibenzoate.

In certain embodiments, the citric acid derivative may be an optionally hydroxylated triester of a C₂-C₈ tricarboxylic acid and of a C₂-C₈ alcohol, such as citric acid esters, such as trioctyl citrate, triethyl citrate, acetyl tributyl citrate, tributyl citrate or acetyl tributyl citrate.

In certain embodiments, the isobutyrate derivative may be a C₁-C₆ carboxylic acid ester of sucrose such as sucrose acetate isobutyrate.

In certain embodiments, the at least one plasticizer may be dipropylene glycol dibenzoate and/or acetyl tributyl citrate.

The at least one plasticizer may be present in an amount ranging from about 0.5%, 2% or 3% to about 3%, 5%, or 10% by weight.

According to embodiments of the present invention, the nail composition also includes at least one solvent. The solvent is generally suitable to dissolve at least 5% or 10% by weight of the water-insoluble cellulose compound and/or the additional film formers at standard conditions. The solvent may be a part of an acetate-rich solvent system. “By acetate-rich solvent system,” it is meant that the proportion of acetate compounds in the solvent system is at least about 30% by weight, such as at least about 50% by weight, such as at least about 70% by weight, such as at least about 85% by weight, such as at least about 90%, where these percentages are relative to all solvents in the composition. Suitable acetate compounds include, for example, C₁-C₄ alkyl acetates such as ethyl acetate, propyl acetate, amyl acetate, and butyl acetate.

The solvents used may have a molecular weight less than or equal to 200. One or more of the solvents (and preferably all of the solvents) may have a boiling point between 55° C. and 250° C. In some embodiments, one or more of the solvents may have a boiling point between 120° C. and 250° C.

The solvent system may include non-acetate compounds that assist in dissolving nitrocellulose and/or the additional film formers. The may include C2-05 monoalcohols such as isopropanol or ethanol. The solvent system may also include C₁-C₄ alkyl lactates or glycol ethers that are capable of dissolving the water-insoluble cellulose compound.

In certain embodiments, the solvent comprises at least one cyclic carbonate ester, including but not limited to, ethylene carbonate and/or propylene carbonate. Furthermore, in certain embodiments, the solvent system includes water. For example, the concentration of water in the composition may be from about 0.1% or 0.5% or 0.75% to about 0.75%, 1% or 2% by weight.

The total amount of solvent system (solvent present in the composition) may range from about 20% or 30% or 40% or 50% or 55% to about 60% or 70% or 80% or 90% by weight.

The composition also includes oils such as dimethicone or alkanes such branched alkanes including isododecane, isohexadecane and the like. The concentration of such compounds if included may range from about 0.1% or 0.2% or 0.5% to about 0.5% or 1% or 2% by weight.

According to certain embodiments of the present application, compositions may optionally include at least colorant. Suitable colorants include, but are not limited to, lipophilic dyes, pigments and pearlescent agents, and their mixtures. Any colorant typically found in nail polish compositions can be used.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescent pigments may also be included, and may be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

If included, the one or more colorants may be present in a total amount from about 0.5% or 1% or 2% to about 3% or 5% or 7% or 10%.

According to certain embodiments, the composition may include other compounds known for nail treatment such as dimethylsulfone (also known as methyl sulphonyl methane). The dimethylsulfone may be present in a concentration such that the weight ratio of urea compound (that is free of both urethane groups and of cyclic groups) to dimethylsulfone ranges from about 1:20 or 1:15 or 1:10 or 1:5 to about 20:1 or 10:1 or 5:1.

The composition may include other optional materials known for use in nail polishes such as fragrances, preservatives, thickeners such as clays, ultraviolet filters, and the like.

Compositions of the present invention may be made using techniques known in the art. For example, any optional pigments may be ground and dispersed in a solvent. Premixes of water-insoluble cellulose polymer or additional film forming polymers may be made by dissolving the polymer in solvent. Ingredients may be combined and mixed under conditions to facilitate full dissolution of the polymers and suspension of any insoluble components.

Compositions of the present invention may be used by applying to the nails such as with a brush or other applicator. A smooth even coating may be applied to fingernails and/or toenails. The coating may be left on for a number of hours or for up to several weeks. According to certain embodiments the composition is applied to the nails and later removed and optionally reapplied and removed a number of times. The inventors have found that compositions of the present invention are capable of strengthening nail and/or reducing surface roughness. Surface roughness may be reduced by more than 10%, such as by more than 15%, such as by more than 20% or even 30%.

Surface roughness may be measured using a Laser Scanning Microscope such as a KEYENCE VR-5000, available from Keyence Corporation of America of Itsaca, Ill. The method of measurement includes placing the testing subject's fingernail on the laser scanning microscope stage and allowing the laser to scan across the fingernail, about 10-20 seconds. The surface roughness is calculated by the software provided.

### EXAMPLE 1

The composition listed in Table 1 was prepared by mixing until homogenous. The mixing continued at an increased mixing speed until homogenous. The list of ingredients is included in Table 1 below. Also included is surface roughness. The untreated nail had a surface roughness of 26.58+/−0.9 microns. While comparative example, Comp. 1 showed only limited improvement in surface roughness, Inventive Examples, Ex. 1 and Ex. 2 showed substantial reduction in surface roughness as compared with the untreated nail.

TABLE 1

| INGREDIENT | Comp. 1 | Ex. 1 | Ex. 2 |
|---|---|---|---|
| Nitrocellulose and | 20-30% | 20-30% | 20-30% |
| Additional Film | | | |
| Formers | | | |
| Plasticizers | 3-8% | 3-8% | 3-8% |
| Acetate-Rich Solvent | 60-75% | 60-75% | 60-75% |
| Blend | | | |
| Hydroxyethyl Urea | 0% | 0.1-1% | 0.1-1% |
| Citric Acid | 0% | 0.1-1% | 0% |
| Other Ingredients | 1-3% | 1-3% | 1-3% |
| SURFACE | 24.05 +/− 2.3 | 22.27 +/− 0.9 | 16.45 +/− 0.5 |
| ROUGHNESS | | | |
| (microns) | | | |

Comparative Example Comp. 1, Inventive Example Ex. 1 and Inventive Example Ex. 2 were identical except that Comp. 1 had no hydroxyethylurea and no citric acid; Ex. 1 had both hydroxyethylurea and citric acid; Ex. 2 had hydroxyethylurea but had no citric acid. Concentrations were all identical. Q.S. was done with ethyl acetate.

### EXAMPLE 2

A composition consistent with embodiments of the invention described herein and consistent in composition with Ex. 1 described above was evaluated for hardness to determine how resistant a material is to various kinds of permanent shape change when a force is applied. Specifically, “Persoz hardness” was determined by applying a thin layer (coating) of the test composition on a glass plate and measuring hardness using a Pendulum Hardness Tester (Persoz pendulum) equipped with a temperature and humidity control chamber (30±2° C., 70±2% RH) according to ASTM method D4366-95. The higher the Persoz number, the harder the layer (coating) is. a glass plate and is then dried at 30° C. for a desired period such as 23 hours (Day 1) or seven days (Day 7), then for one additional hour in the environment of the pendulum. The Persoz pendulum was then positioned above the plate. The time required by the Persoz pendulum to oscillate from an amplitude of 12° to an amplitude of 4° was then determined. A plurality of measurements, e.g. ten measurements, can be taken so as to establish an average of the calculated results. The results indicate that the Day 1 hardness was 27 and the Day 7 hardness was 38.

### EXAMPLE 3

The inventive Example noted above in Example 2 was evaluated for moisturizing effects, as compared with a similar formula having no Hydroxyethyl Urea and no citric acid as well as an uncoated substrate. The test was performed by taking a VITRO-NAILS substrate (available from Florida Suncare Testing, Inc., IMS Division, Bunnell, Fla.) and dip coating with a test composition. The samples are allowed to dry for at least eight hours. The sample is then placed in a humidity chamber at 37C/50% RH for various time periods such as 50 minutes. Samples are removed from the humidity chamber and allowed to equilibrate to ambient conditions. Water absorption is calculated by measuring the gain in mass compared with prior to entry into the humidity chamber. At 50 minutes, the water absorption for uncoated VITRO NAIL was 0.0001 mg/mg, and the water absorption for the composition without the hydroxyethyl urea and citric acid was 0.0195 mg/mg, whereas the water absorption for the composition with the hydroxyethyl urea and citric acid was 0.0341 mg/mg.

Those skilled in the art will recognize or be able to ascertain using no more than routine experimentation, many equivalents to the specific embodiments of the invention described herein. Such equivalents are intended to be encompassed by the following claims:

What is claimed is:

1. A cosmetic composition for treating the nails, wherein the cosmetic composition comprises:
a water-insoluble cellulose polymer;
one or more additional film-formers;
plasticizer;
a urea compound free of both urethane groups and of cyclic groups; and wherein the cosmetic composition is free of urea compounds having either or both of urethane groups and cyclic groups, and wherein the cosmetic composition is anhydrous.

2. The cosmetic composition of claim 1, wherein the urea compound free of both urethane groups and of cyclic groups is water-soluble.

3. The cosmetic composition of claim 1, wherein the urea compound free of both urethane groups and of cyclic groups is a hydroxyalkylurea.

4. The cosmetic composition of claim 1, wherein the water-insoluble cellulose polymer is nitrocellulose.

5. The cosmetic composition of claim 1, further comprising an acetate-rich solvent system.

6. The cosmetic composition of claim 1, further comprising dimethyl sulfone.

7. A cosmetic composition for treating the nails, wherein the cosmetic composition comprising:
from about 5% by weight to about 20% by weight of nitrocellulose;
from about 5% by weight to about 20% by weight of one or more additional film-formers;
plasticizer;
from about 50% to about 90% by weight of an acetate-rich solvent system; and
a urea compound free of both urethane groups and of cyclic groups; wherein the nitrocellulose and the one or more one or more additional film-formers form a film-forming system, wherein the ratio by weight of the urea compound free of both urethane groups and of cyclic groups to the film-forming system is from about 1:500 to about 1:50, and wherein the cosmetic composition is free of urea compounds having either or both of urethane groups and cyclic groups, and wherein the cosmetic composition is anhydrous.

8. The cosmetic composition of claim 1, wherein the cosmetic composition is substantially free of pigment.

9. A cosmetic composition for treating the nails, wherein the cosmetic composition comprises:
a water-insoluble cellulose polymer;
one or more additional film-formers;
plasticizer;
a urea compound; and
one or more organic polycarboxylic acids, wherein the cosmetic composition is anhydrous.

10. The cosmetic composition of claim 9, wherein the urea compound is free of both urethane groups and of cyclic groups.

11. The cosmetic composition of claim 9, wherein the urea compound is water-soluble.

12. The cosmetic composition of claim 9, wherein the urea compound is a hydroxyalkylurea.

13. The cosmetic composition of claim 9, wherein the water-insoluble cellulose polymer is nitrocellulose.

14. The cosmetic composition of claim 9, further comprising an acetate-rich solvent system.

15. The cosmetic composition of claim 1, further comprising dimethyl sulfone.

16. The cosmetic composition of claim 1, wherein the urea compound free of both urethane groups and of cyclic groups is hydroxyethylurea and the one or more organic polycarboxylic acids comprises citric acid.

17. The cosmetic composition of claim 16, wherein the citric acid and hydroxyethylurea are present in a ratio by weight that is ratio from about 10:1 to about 2:10.

18. A method of treating the nails, comprising applying to said nails a cosmetic composition comprising:
a water-insoluble cellulose polymer;
one or more additional film-formers;
plasticizer; and
a urea compound free of both urethane groups and of cyclic groups; wherein the cosmetic composition is free of urea compounds having either or both of urethane groups and cyclic groups, and wherein the cosmetic composition is anhydrous.

19. The method of claim 18, wherein the water-insoluble cellulose polymer is nitrocellulose, and wherein urea compound free of both urethane groups and of cyclic groups is a hydroxyalkylurea, wherein the nitrocellulose and the one or more one or more additional film-formers form a film-forming system, wherein the ratio by weight of the urea compound free of both urethane groups and of cyclic groups to the film-forming system is from about 1:500 to about 1:50.

20. The method of claim 18, further comprising removing the cosmetic composition, and optionally reapplying and removing the composition one or more times to effect a reduction in surface roughness in the nail of greater than 10%.
